(19) United States
(12) Patent Application Publication
(10) Pub. No.: US 2020/0216863 A1
(43) Pub. Date: Jul. 9, 2020

(54) Use of renewable deep eutectic solvents in a one-pot process for a biomass

(71) Applicants: National Technology & Engineering Solutions of Sandia, LLC, Albuquerque, NM (US); The Regents of the University of California, Oakland, CA (US)

(72) Inventors: Feng Xu, Albany, CA (US); Blake A. Simmons, San Francisco, CA (US); Seema Singh, Clarksburg, MD (US)

(73) Assignees: National Technology & Engineering Solutions of Sandia, LLC, Albuquerque, NM (US); The Regents of the University of California, Oakland, CA (US)

(21) Appl. No.: 16/737,724

(22) Filed: Jan. 8, 2020

Related U.S. Application Data
(60) Provisional application No. 62/789,960, filed on Jan. 8, 2019.

(51) Int. Cl. C12P 7/06; C12P 19/02; C12P 19/14

(52) U.S. Cl. CPC C12P 7/06; C12P 2201/00; C12P 19/14; C12P 19/02

(57) ABSTRACT

The present invention provides for a method to produce a biofuel and/or chemical compound from a biomass, the method comprising: (a) introducing a biomass and a deep eutectic solvent (DES), or mixture thereof, into a vessel to form a one-pot composition, wherein the DES, or mixture thereof, solubilizes the biomass; (b) introducing an enzyme and/or a microbe to the one-pot composition such that the enzyme and/or microbe produce a biofuel and/or chemical compound from the solubilized biomass; and, (c) optionally the biofuel and/or chemical compound is separated from the one-pot composition.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of using deep eutectic solvents for biomass pretreatment.

### BACKGROUND OF THE INVENTION

Biofuels and bioproducts derived from sustainable feedstocks are considered a potential solution to address the challenges associated with human population growth.(1) For efficient biofuel production, the biochemical conversion of lignocellulosic biomass has been frequently discussed in terms of process optimization as well as the reaction mechanism of various thermochemical processing (e.g., pretreatment) and biochemical conversion (e.g., enzymatic hydrolysis and fermentation).(2-4) Current challenges to the realization of an affordable and scalable biomass conversion technology are those associated with complicated process designs, difficulties associated with efficient solvent recycle, and water consumption.(5,6)

Process intensification by minimizing separations has the potential to significantly reduce energy and water usage. Process intensification and integration within a biorefinery context can be challenging because of the typical discrepancy between the conditions used for pretreatment and those used downstream for saccharification and fermentation. For example, pretreatment usually employs acidic or basic conditions to disrupt the lignocellulosic plant cell wall and/or decrystallize cellulose for improved enzyme accessibility. Reagents used in pretreatment are usually not compatible with downstream processing (e.g., enzymatic saccharification and microbial fermentation) because of the differences in pH optima or the toxicity of the reagents and byproducts from the pretreatment process. The recent advent of a certain class of biocompatible ionic liquids (ILs, such as cholinium lysinate) presents a very promising development in pretreatment due to their effectiveness and low toxicity to the enzymes or microbes after pH adjustment.(5,6) These features allow for a consolidated one-pot biomass-to-biofuel conversion process that combines pretreatment, saccharification, and fermentation in one vessel,(5,6) but the process requires the use of additional reagents and steps that can be challenging, especially in a high biomass loading process. To overcome these challenges, there is a need to identify alternative solvent systems that do not require pH adjustment after pretreatment.

One example of an alternative IL is found in the use of certain protic alkylammonium ILs that do not require pH adjustments, water-washes, and solid-liquid separations after pretreatment.(7) However, these protic ILs were toxic to the microbes used in biofuel production and required significant dilution of the pretreatment effluent to reach acceptable concentrations. There has been recent attention on the use of certain deep eutectic solvents (DESs)(8-14) because they share many characteristics and properties with ILs,(15,16) and can be powerful lignocellulosic biomass pretreatment solvents.(17,18) DESs are usually prepared by mixing a hydrogen bonding donor (HBD) with a salt, a relatively convenient and inexpensive process as compared to the synthesis of most conventional ILs.(16,19) The numerous combinations of DES precursors also provide an opportunity to identify a biocompatible DES that is also effective at biomass pretreatment. To the best of our knowledge, there have been no reports describing the use of DESs in a consolidated biofuel production process.

### SUMMARY OF THE INVENTION

The present invention provides for a method to produce a biofuel and/or chemical compound from a biomass, the method comprising: (a) introducing a biomass and a deep eutectic solvent (DES), or mixture thereof, into a vessel to form a one-pot composition, wherein the DES, or mixture thereof, solubilizes the biomass; (b) introducing an enzyme and/or a microbe to the one-pot composition such that the enzyme and/or microbe produce a biofuel and/or chemical compound from the solubilized biomass; and, (c) optionally separating the biofuel and/or chemical compound from the one-pot composition. In some embodiments, the introducing steps (a) and (b), and optionally the separating step (c), are continuous.

The method, or one-pot method, does not require any solid-liquid separation step. The one-pot method does not require adjustment of the pH level in the one-pot composition. The one-pot method does not require any dilution, or addition of water or medium, after pretreatment and/or before saccharification and fermentation. The reaction of the enzyme and the growth of the microbe occur in the same one-pot composition. The DES, or mixture thereof, is renewable as it can be continuous in use. The one-pot method can produce a yield of sugar that is equal to or more than about 70%, 75%, or 80%, or any other value described herein.

The present invention also provides for one-pot compositions described herein.

The present invention also provides for a mixture of DESs produced by the method described herein.

The present invention provides for a method for producing renewable fuel or chemical using a one-pot process approach from cellulosic biomass. The present invention also provides for the use of renewable solvents that are compatible with both enzymes, such as a cellulase, and microbes, such as yeast. The present invention also provides for the use of a DES mixture that comprises choline chloride and glycerol with a molar ratio of about 1:2.

The one-pot biomass pretreatment, saccharification, and fermentation with bio-compatible deep eutectic solvents (DESs). The used bio-compatible DESs are tested for microbial, such as yeast, compatibility and toxicity. The pretreatment efficacy of the selected DESs are tested. The uses of the DESs for biomass processing eliminates the need to remove any solvent after biomass pretreatment, thus making the one-pot approach possible.

Using bio-compatible DESs enables a one-pot biomass conversion which eliminates the needs of mass transfer between reactors and the separation of solid and liquid. In some embodiments, the method does not require recycling any catalyst and/or enzyme. In some embodiments, the method requires less water usage than current biomass pretreatment. The method can produce fuels/chemicals at a higher titer and/or yield in a single vessel without any need for intermediate units of mass transfer and/or solid/liquid separation.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Plots of S. cerevisiae BY4741 growth in the presence of 5 wt % DESs in aqueous solutions: Ch1, [Ch][Cl]-2urea; Ch2, [Ch][Cl]-oxalic acid; Ch5, [Ch][Cl]-2ethylene glycol; Ch6, [Ch][Cl]-3ethylene glycol; Ch8, [Ch][Cl]-2levulinic acid; Ch9, [Ch][Cl]-xylitol; Ch10, [Ch][Cl]-D-sorbitol; Ch11, [Ch][Cl]-2D-isosorbide; Ch12, [Ch][Cl]-2glycerol.

FIG. 2A. Plot of sugar yields as a function of DES at pretreatment conditions of 180° C. and 2 h followed by saccharification.

FIG. 2B. Lignin extraction and glucose yield using a one-pot approach using DES-Ch12 (AIL: acid insoluble lignin).

FIG. 3A. Sugar degradation compounds from corn stover pretreatment with DES-Ch12 (HMF: hydroxymethylfurfural).

FIG. 3B. The phenolic compounds from lignin degradation after DES-Ch12 pretreatment.

FIG. 4A. One-pot bioethanol production with biocompatible deep eutectic solvents (DES-Ch12).

FIG. 4B. Glucose balance of the one-pot bioethanol conversion.

FIG. 5A. Yeast growth profile with different DESs in different concentrations. A: Ch1 as a mixture of [Ch][Cl] and urea (2:1).

FIG. 5B. Yeast growth profile with different DESs in different concentrations. B: Ch5 as a mixture of [Ch][Cl] and ethylene glycol (2:1).

FIG. 5C. Yeast growth profile with different DESs in different concentrations. C: Ch6 as a mixture of [Ch][Cl] and ethylene glycol (3:1).

FIG. 5D. Yeast growth profile with different DESs in different concentrations. D: Ch9 as a mixture of [Ch][Cl] and xylitol (1:1).

FIG. 5E. Yeast growth profile with different DESs in different concentrations. E: Ch11 as a mixture of [Ch][Cl] and D-Isosorbide (2:1).

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

DESs are systems formed from a eutectic mixture of Lewis or Brønsted acids and bases which can contain a variety of anionic and/or cationic species. DESs can form a eutectic point in a two-component phase system. DESs are formed by complexation of quaternary ammonium salts (such as, choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like.

Typically, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100° C. with specific molar ratio with choline chloride. This class of DES does not require a multistep synthesis. The novel DES is synthesized from lignin which is a renewable source.

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes.

The DES, or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme's enzymatic activity and/or the microbe's growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the introducing steps (a) and (b) together produce a sugar and a lignin from the biomass. The lignin can further be processed to produce a DES. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the steps (a) to (c) are continuous, the one-pot composition is in a steady state.

In some embodiments, the DES is one taught in WO 2018/204424 (Seem Singh et al.), which is hereby incorporated in its entirety by reference. In some embodiments, the DES is synthesized from a lignin derived monomeric phenol. In some embodiments, the DES is synthesized from a lignin derived monomeric phenol derived from the biomass. In some embodiments, the lignin derived monomeric phenol is:

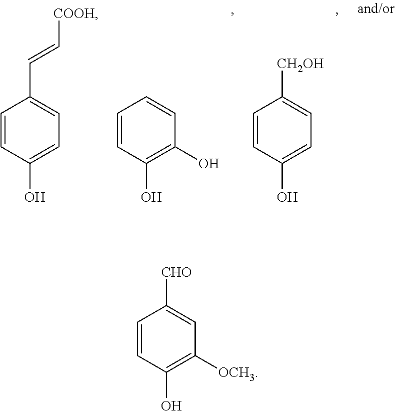 [SMILES: O=C(O)/C=C/c1ccc(O)cc1 ; Oc1ccccc1O ; "," ; OCc1ccc(O)cc1 ; "," ; COc1cc(C=O)ccc1O]

In some embodiments, all or some of the one-pot composition is further pretreated as follows: the method further comprising: (d) optionally separating the sugar and the lignin in the one-pot composition, (e) depolymerizing and/or converting the lignin into one or more lignin derived monomeric phenol, or a mixture thereof, (f) providing the one or more lignin derived monomeric phenol, or a mixture thereof, in a solution, (g) introducing one or more quaternary ammonium salts, or a mixture thereof, to the solution, (h) heating the solution, such that steps (g) and (h) together result in the synthesis of a DES, (i) optionally forming a DES system from the DES synthesized in step (h), and (j) optionally repeating steps (d) to (i) using the DES system formed in step (i) in the introducing step (a).

In some embodiments, the heating step (h) comprises increasing the temperature of the solution to a value within a range of about 75° C. to about 125° C. In some embodiments, the heating step (h) comprises increasing the temperature of the solution to a value within a range of about 80° C. to about 120° C. In some embodiments, the heating step (h) comprises increasing the temperature of the solution to a value within a range of about 90° C. to about 110° C. In some embodiments, the heating step (h) comprises increasing the temperature of the solution to about 100° C.

In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/U52011/053787, PCT/U52011/058660, PCT/U52011/059784, PCT/U52011/061900, PCT/U52012/031025, and PCT/US2013/074214 (all of which are incorporated in their entireties by reference).

In some embodiments, the biofuel produced is ethanol, or any other organic molecule, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Deep eutectic solvents (DESs) share the promising solvent properties of ionic liquids. They show low volatility, wide liquid range, water-compatibility, non-flammability, non-toxicity, biocompatibility and biodegradability. Furthermore, DES can be easily prepared from readily available materials at high purities and low cost compared to ILs. Lignin is the second most abundant naturally occurring polymer next to cellulose, which represents a significant component of carbon on earth. Large amount of technical lignins such as Kraft lignin and lignosulfonate is produced as by-products in the pulp and paper industries. It is also expected that more lignin will become available in coming years as the production capability of second generation of biofuels increases. As a renewable and resource, lignin and lignin derived products (phenolic) are an important material. DESs with lignin-derived phenolic compounds either as a single monomer or phenolic mixture can be used in the present invention.

The one-pot biomass pretreatment, saccharification, and fermentation with bio-compatible deep eutectic solvents (DESs). The used bio-compatible DESs are tested for microbial, such as yeast, compatibility and toxicity. The pretreatment efficacy of the selected DESs are tested. The uses of the DESs for biomass processing eliminates the need to remove any solvent after biomass pretreatment, thus making the one-pot approach possible.

In some embodiments, the biomass is a lignocellulosic biomass. In some embodiments, the vessel is made of a material that is inert, such as stainless steel or glass, that does not react or interfere with the reactions in the one-pot composition.

In some embodiments, the pretreatment comprises about 0.5 g of biomass (such as corn stover) mixed thoroughly with about 4.5 g DES (such as choline chloride and glycerol) in a suitable inert vessel, such as a glass vessel, followed by heating up to about 180° C. and the temperature is maintained for a suitable period of time, such as about 2 hours. After pretreatment, the resulting slurry is cooled to below about 50° C. and is immediately ready for the following saccharification and microbial conversion. The saccharification is carried out with a suitable enzyme, such as a commercial enzyme mixture (for example, CTec2 and HTec2 from Novozymes A/S (Bagsværd, Denmark), at about 50° C. and about pH 5 at about 48 rpm in an incubator with shaking function. After a suitable period of time, such as about 48 hours, of saccharification, the generated sugar stream is then immediately ready for microbial conversion. For example, a wild-type yeast, such as Saccharomyces cerevisiae, is inoculated at temperature (about 30° C. to about 37° C.) for anaerobic ethanol fermentation.

Using bio-compatible DESs enables a one-pot biomass conversion which eliminates the needs of mass transfer between reactors and the separation of solid and liquid. In some embodiments, the method does not require recycling any catalyst and/or enzyme. In some embodiments, the method requires less water usage than current biomass pretreatment. The method can produce fuels/chemicals at a higher titer and/or yield in a single vessel without any need for intermediate units of mass transfer and/or solid/liquid separation.

### EXAMPLE 1

### Biocompatible Choline-Based Deep Eutectic Solvents Enable One-Pot Production of Cellulosic Ethanol

Previous configurations of biomass conversion technologies based on the use of ionic liquids (ILs) suffer from problems such as high operating costs and large amounts of water used. There have been recent efforts toward process intensification and integration to realize a one-pot approach for biofuel production using certain ILs, but these typically still require pH adjustment and/or dilution after pretreatment and before saccharification and fermentation. Deep eutectic solvents (DESs) were investigated as an alternative to ILs to address these challenges, and the results obtained suggest that certain DESs are compatible with hydrolytic enzymes and common biofuel producing microorganisms such as Saccharomyces cerevisiae. Among the DESs investigated, choline chloride/glycerol (Ch12) achieved the highest rates of lignin extraction and pretreatment efficiency in terms of sugar yields (>80%) after enzymatic hydrolysis. Most importantly, the DES-Ch12-based “one-pot” biomass conversion process does not require any pH adjustment before commencing with saccharification and fermentation. Degradation compounds generated from polysaccharides (e.g., furfural) and lignin (e.g., ferulic acid) during biomass conversion were characterized and evaluated for their potential inhibitory effect on yeast growth and biofuel production. We conclude that this DES can be used to achieve biofuel (e.g., ethanol) production with a theoretical yield of 77.5% based on the initial glucan present in the biomass in a consolidated one-pot process configuration, redefining biomass conversion using DESs.

This work introduces a set of biocompatible DESs that appear promising for use in the conversion of biomass into biofuels and bioproducts using a one-pot process. Since choline chloride is a relatively inexpensive, biodegradable, and nontoxic compound that can be extracted from biomass or readily synthesized from fossil reserves,(15) it was used as an organic salt to produce DESs. The mechanism of biomass pretreatment in the presence of the synthesized DESs was identified, and the processing conditions were optimized to decrease energy inputs and time. Potential cytotoxic byproducts, such as furfurals, that may be formed during biomass pretreatment were monitored, and their impact on saccharification and fermentation is evaluated and discussed. To our knowledge, this is the first report that uses a biocompatible DES that integrates biomass pretreatment, saccharification, and fermentation in a single vessel without any solid/liquid separation and/or pH adjustment.

### Material and Methods

All of the chemicals were reagent grade and purchased from Sigma-Aldrich (St. Louis, Mo.) if not specified otherwise. Corn stover was supplied by Michigan State University and prepared as reported.(6) The enzymes (Cellic CTec 2 and HTec 2) were a gift from Novozymes North America (Franklinton, N.C.), containing 188 mg protein per mL.

### DESs Preparation

Choline chloride and nine hydrogen bond donor molecules were mixed in the ratios listed in Table 1. The mixture was heated and stirred at 30, 60, or 80° C. in a conical flask with plug to reduce volatilization until a homogeneous colorless liquid was formed. Afterward, the synthesized DESs were kept in a vacuum desiccator with silica gel until further use.

TABLE 1
Selected Choline-Chloride-Based Room Temperature
Deep Eutectic Solvents (DES).

| | | | molar ratio of | |
|---|---|---|---|---|
| DES | halide salt | HBD^{a} | HBD to salt | pH^{b} |
| Ch1 | [Ch][Cl] | urea | 2 | 9.5 |
| Ch2 | [Ch][Cl] | oxalic acid | 1 | 0.7 |
| Ch5 | [Ch][Cl] | ethylene glycol | 2 | 4.3 |
| Ch6 | [Ch][Cl] | ethylene glycol | 3 | 4.8 |
| Ch8 | [Ch][Cl] | levulinic acid | 2 | 2.2 |
| Ch9 | [Ch][Cl] | xylitol | 1 | 4.5 |
| Ch10 | [Ch][Cl] | _{D}-sorbitol | 1 | 4.9 |
| Ch11 | [Ch][Cl] | _{D}-isosorbide | 2 | 4.2 |
| Ch12 | [Ch][Cl] | glycerol | 2 | 5.8 |
| | [Ch][Cl] | | | 5.3 |

^{a}HBD: hydrogen bond donor.
^{b}The pH of DES was measured at a 10% aqueous solution.

### Biomass Pretreatment

A corn stover solid loading of 10% was used. For example, 0.5 g of corn stover was mixed thoroughly with 4.5 g of DES in a pressure tube (50 mL, Ace Glass Inc., Vineland, N.J.), and the tube was then heated in an oil bath at a certain temperature for a few hours. The pretreated biomass was separated by centrifugation for compositional analysis. Briefly, the pretreated biomass was washed by deionized water at least three times, and the solid fraction was collected after each wash by centrifugation. The solid fraction was then lyophilized for composition. The composition analysis was conducted according to the published NREL procedure.(20)

### Enzymatic Saccharification

The digestibility test was conducted with either a one-pot approach or a conventional approach in which the solid fraction was washed and separated before saccharification. Particularly, in a one-pot process of saccharification, 1 M citric acid/citrate buffer was added to the pretreated biomass slurry for a final buffer concentration of 50 mM. The mixture of DES and biomass (e.g., 40 mL, in which DES is 4.5 g, corn stover is 0.5 g) was then tested for sugar yield in a 50 mL screwcap Falcon tube. The saccharification was carried out at 50° C. for 3 days (saccharification only) and pH 5 at 48 rpm in a rotary incubator (Enviro-Genie, Scientific Industries, Inc.) using commercial enzyme mixtures, Cellic CTec2 and HTec2, with an enzyme dosage of 20 mg protein per gram glucan and 2 mg protein per gram xylan, respectively.

### Fermentation

For ethanol production assays, Saccharomyces cerevisiae strain BY4741 (MATa his3Δ0 leu2Δ0 met15Δ0 ura3Δ0), a derivative of S288C, was cultivated according to the published NREL procedure.(20) Yeast was inoculated directly into concentrated hydrolysates from saccharification.(6) For an integrated one-pot ethanol SSF, the temperature was decreased after a 1 day presaccharification (50° C.), and the SSF was then conducted with yeast loading of 3 g/L (based on cell weight) under fermentative conditions at 120 rpm at 37° C. for 2-3 days.

### Analysis of Sugars, Ethanol, and Other Degradation Compounds

The concentration of sugar, ethanol, HMF, and furfural was measured by HPLC (Agilent HPLC 1200 Series) equipped with a Bio-Rad Aminex HPX-87H column and a refractive index detector. The solid fraction after saccharification or fermentation in a dilute solution is below 1 wt % after dilution, and its volume displacement could then be negligible. Glucose yield and ethanol yield were calculated on the basis of the glucan content in corn stover, as 1.11 g glucose per gram glucan and 0.568 g ethanol per gram glucan, respectively. The phenolic compounds derived from lignin were determined using an LC-MSD according to the previously reported method.(21)

### Results and Discussion

### DESs as Biocompatible Solvents for Biomass Conversion

One-pot biomass conversion can reduce the operating costs of biofuel production because it simplifies process design and reduces the energy input for the mass transfer between reactors that is typically required in a traditional biomass process.(6,9) One of the key elements for a successful one-pot process is the use of biocompatible reagents at all steps of the process, and it is important to screen DESs and determine their relative biocompatibility. Choline-based DESs are promising in this regard because they are bioderived and can be produced in bulk.(17) All DESs prepared in this study are liquids at room temperature. A number of DES candidates based on [Ch][Cl] and various HBDs were selected and mixed in a certain molar ratio of HBD to salt (Table 1). Another key element for the successful consolidation process is a suitable pH value of DES that could enable downstream bioconversion without pH adjustment.(5-7) Table 1 shows that the measured pH values of the as-synthesized DESs in their 10 wt % aqueous solutions vary in a broad range from 0.7 to 9.5. These varieties of pH values are mainly ascribed to the nature of HBD since [Ch][Cl] solution is a weak acidic salt with a pH value of around 5.3 in a 10 wt % concentration.

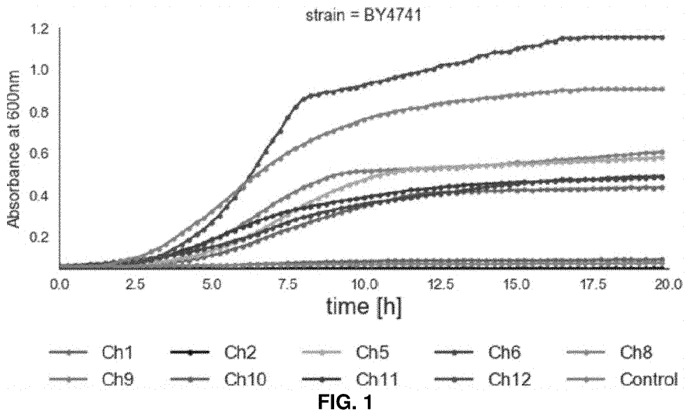

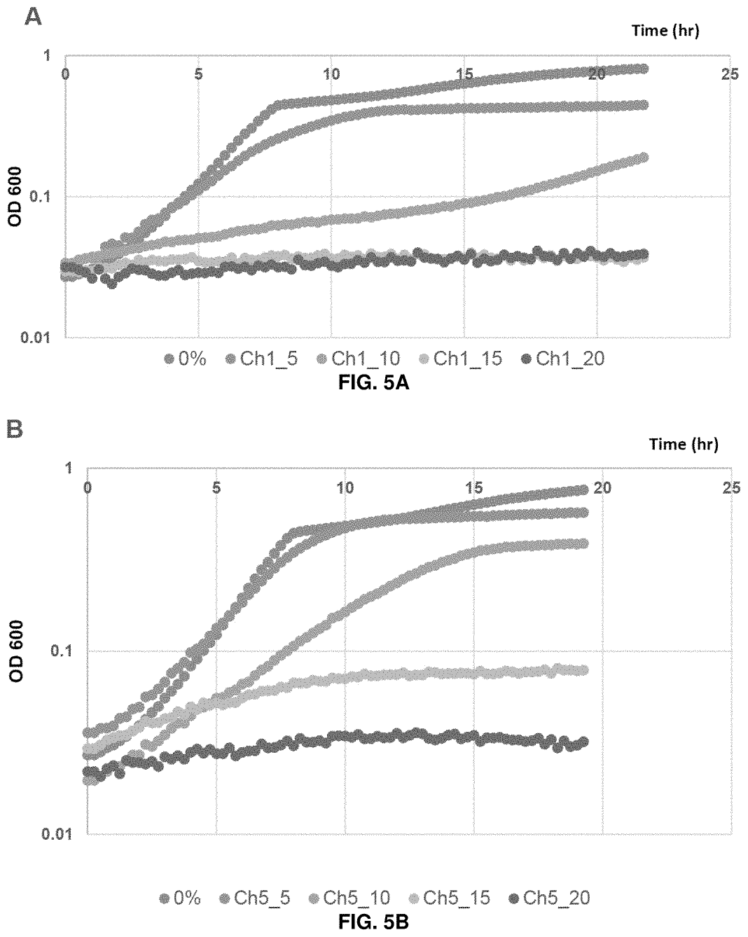

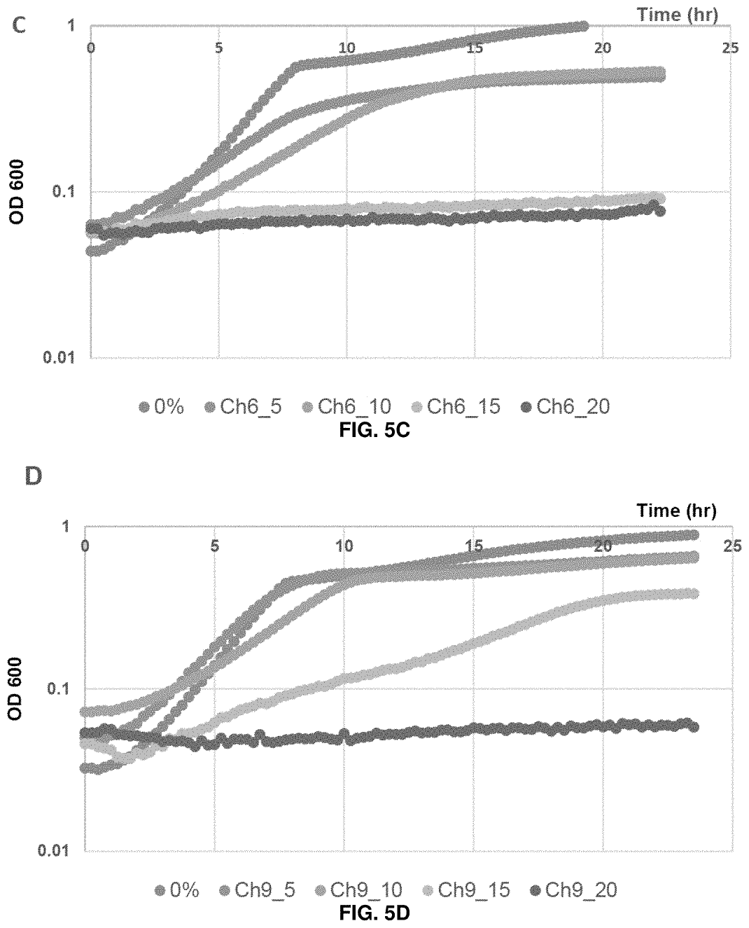

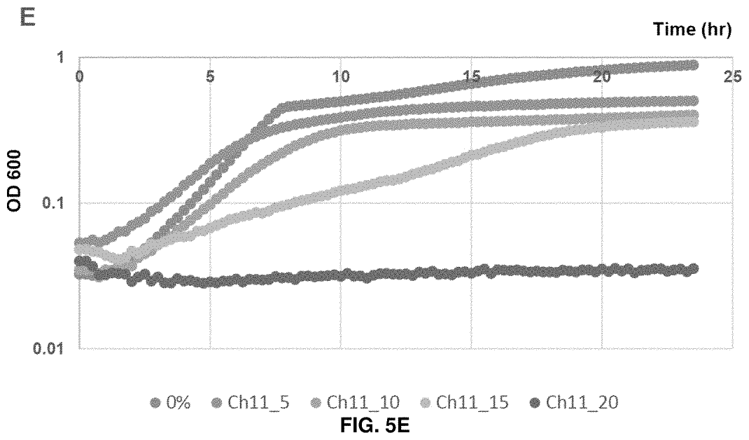

Saccharomyces cerevisiae is a well-studied and established host for the industrial production of ethanol,(22) and was thus employed as production host in this study. FIG. 1 shows the S. cerevisiae BY4741 growth profile in the presence of 5 wt % of various DES aqueous solutions. Six DES candidates were prepared by mixing [Ch][Cl] with urea, ethylene glycol, xylitol, isosorbide, and glycerol in different molar ratios (abbreviated as Ch1, Ch5, Ch6, Ch9, Ch11, and Ch12, respectively), and were identified as promising in terms of biocompatibility, with yeast growth reaching similar cell density as those grown without DESs present (FIG. 1). In particular the DES-Ch12 showed excellent biocompatibility (FIG. 1). FIGS. 5A to 5E further show the growth profiles of the yeast strain in the presence of some of the synthesized DESs at different concentrations.

### Impact of Delignification on Sugar Production

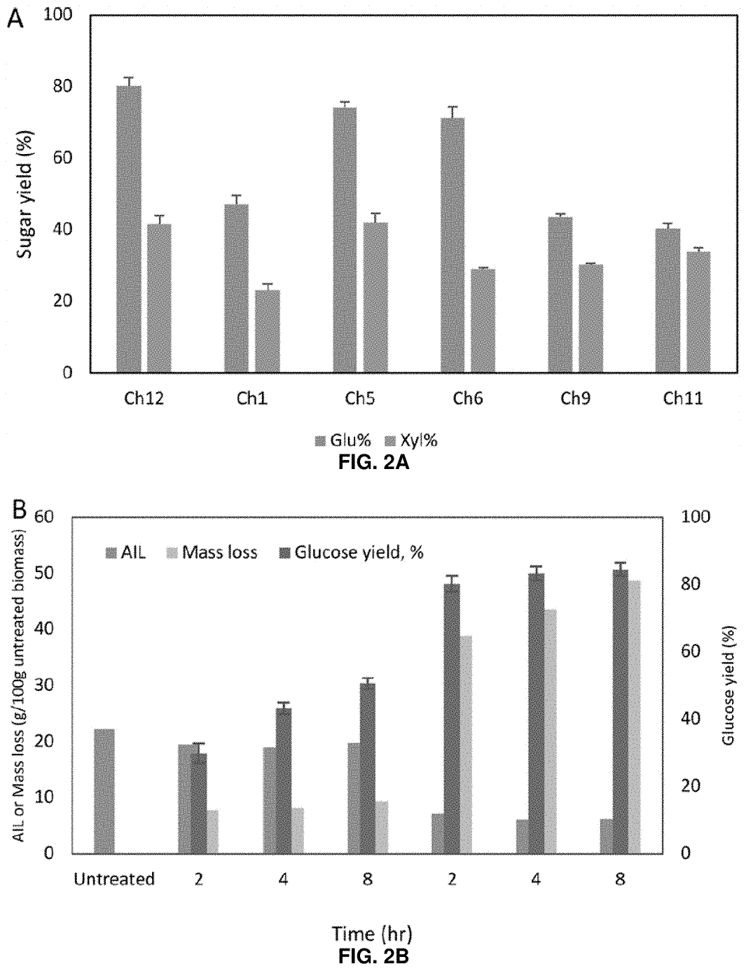

The selected biocompatible DESs were further investigated in terms of biomass pretreatment efficiency as measured by sugar yield after saccharification, as well as lignin extraction efficiency, under a variety of processing conditions. FIGS. 2A and 2B show the effect of selected process conditions on sugar yield and mass loss. As shown in FIG. 2A, the selected biocompatible DESs showed different yields of sugar production, with DES-Ch5, -Ch6, and -Ch12 generating >70% yields following pretreatment and saccharification. The xylose yields are relatively low (below 50%), compared to other biomass processing methods.(23) Compositional analysis indicates that 30-40% of xylan is hydrolyzed into the liquid phase after pretreatment, resulting in a significant mass loss.(24)

On the basis of these initial results, we then studied the performance of DES-Ch12 using different combinations of process temperatures and pretreatment times. As shown in FIG. 2A, glucose yields increased with increases in either temperature or time and are generally attributed to increased delignification, which increases the accessible area of polysaccharides and reduces the absorption of enzymes by lignin.(25) The results show that a significant portion (>60%) of lignin was removed at 180° C., whereas the lignin extraction was ˜10% at 160° C. (FIG. 2B). The significant improvement in lignin removal with an increase of temperature is consistent with previous studies,(7,26,27) and is most likely ascribed to the enhanced cleavage of ether bonds of lignin facilitating lignin extraction from the biomass under higher temperature.(27) The change in pretreatment time, however, does not significantly affect delignification. At 160° C., the glucose yields increased significantly with increases in time despite minimal delignification, and this is attributed to expansion of the cellulose fibers through DES penetration into the fiber bundles.(28)

### Tracking the Formation and Impact of Inhibitory Compounds

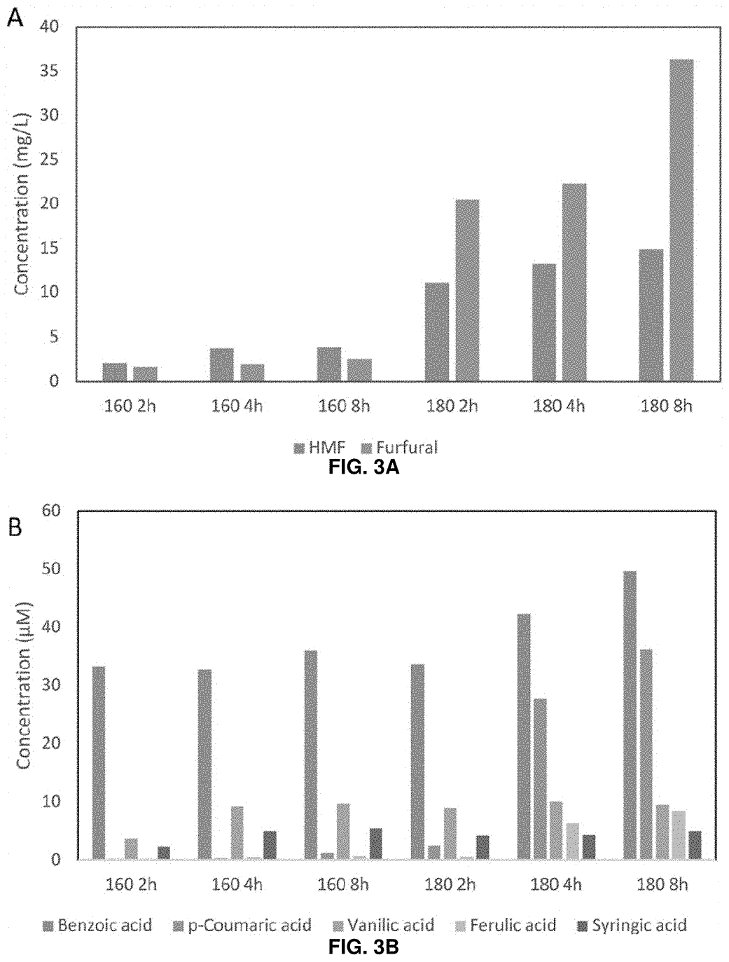

It is known that sugars can generate inhibitory compounds such as hydroxymethylfurfural (HMF) and furfural at high pretreatment temperatures and are considered toxic to the yeast at certain concentrations.(21) The reported inhibition of HMF for yeast growth is significant at 3 g L⁻¹ and decreases ethanol yield from 99% to 89%.(29) We monitored the production of these compounds during DES pretreatment by using high-performance liquid chromatography (HPLC), and the results are shown in FIGS. 3A and 3B. The concentrations of HMF (e.g., 14.9 mg L⁻¹) and furfural (e.g., 36.4 mg L⁻¹) are significantly below the reported inhibitory concentrations for yeast fermentation. The results suggest that the DES process can provide hydrolysates with minimal inhibition of yeast growth and biofuel production.

As DES-Ch12 showed high levels of delignification, it is expected that inhibitory phenolic compounds such as p-coumaric acid and ferulic acid might be formed, and the hydrolysates were analyzed using liquid chromatography-mass selective detector (LC-MSD).(18) An increase in pretreatment severity did increase the concentration of the phenolic compounds generated, and this finding is consistent with increased lignin extraction efficiency (FIG. 3A). Benzoic acid and p-coumaric acid are the dominant lignin degradation compounds detected in the hydrolysates, but their concentrations are very low and are not above the ˜1 mM required for inhibition of yeast growth (FIG. 3B).

### One-Pot Biomass Conversion to Biofuel

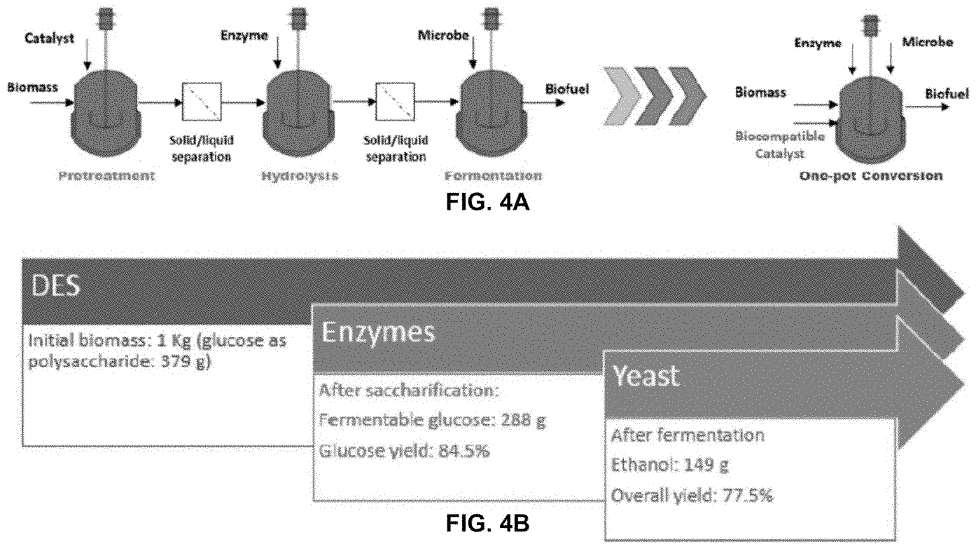

A two-stage temperature controlling strategy was employed for saccharification and fermentation, based on our previous configuration for ethanol fermentation with bionic liquids.(6) Presaccharification of the pretreated slurry was first conducted at 50° C. for 24 h, and then followed by a simultaneous saccharification and yeast fermentation at 37° C. for 48 h. A multistep ethanol conversion from corn stover was then successfully demonstrated in a single vessel. Compared to conventional configurations, the one-pot process with DES-Ch12 eliminated all solid/liquid separation steps (FIGS. 4A and 4B) and did not require any pH adjustment. The process generated 134 g of ethanol from 1 kg of corn stover, which is equal to a conversion yield of 77.5% based on the glucose present.

### Feasibility of C6-C5 Sugar Cofermentation

Next, in order to test the feasibility of C5/C6 sugar cofermentation and thus to further improve the economic feasibility of the process,(22) the yeast strain JBEI-9009, developed in our lab for optimized xylose consumption,(30) was used for further experiments.

The effect of DES-Ch12 supplementation on growth and carbon utilization of JBEI-9009 was analyzed in a plate reader-based assay. JBEI-9009 as well as the unmodified control strain can tolerate 10 wt % Ch12 in rich media (YPD) without showing significant growth limitations. To test if the presence of DES-Ch12 in the media impairs the ability of the strain JBEI-9009 to efficiently utilize xylose, the strain was cultivated in CSM media with 2 wt % xylose containing 10 wt % Ch12 and final samples were taken for HPLC measurements. It was observed that, even though the strain exhibits a longer lag phase when cultivated in the presence of DES-Ch12, it eventually reaches a similar final OD600 and shows similar consumption of xylose in the media.

### CONCLUSIONS

Biocompatible DESs were prepared by mixing choline chloride and a range of salts. Some of the DESs studied were demonstrated to be effective biomass pretreatment solvents and were found to be biocompatible and did not inhibit yeast growth. The generation of inhibitory degradation compounds from polysaccharides and lignin during pretreatment was also monitored, and the levels of these compounds detected were below reported toxicity thresholds. DES-Ch12 was demonstrated to be an effective pretreatment solvent that enabled the consolidation of saccharification and fermentation into a one-pot process that generated high yields of ethanol from corn stover. This promising approach offers significant advantages over other IL and DES biomass conversion technologies in that it does not require pH adjustment or dilution between pretreatment, saccharification, and fermentation unit operations. In addition, the use of inexpensive renewable chemicals as the precursors for DESs may minimize the operational costs and environmental footprint of the entire process, providing a more affordable, sustainable, and scalable biorefinery. Future work should be directed at the development of biofuel hosts that can convert all types of sugars produced, and/or the conversion of engineered bioenergy crops with enhanced C6 content, using this DES one-pot process.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to produce a biofuel and/or chemical compound from a biomass, the method comprising:
(a) introducing a biomass and a deep eutectic solvent (DES), or mixture thereof, into a vessel to form a one-pot composition, wherein the DES, or mixture thereof, solubilizes the biomass;
(b) introducing an enzyme and/or a microbe to the one-pot composition such that the enzyme and/or microbe produce a biofuel and/or chemical compound from the solubilized biomass; and,
(c) optionally the biofuel and/or chemical compound is separated from the one-pot composition.

2. The method of claim 1, wherein the DES comprises a halide salt and a hydrogen bond donor (HBD).

3. The method of claim 2, wherein the halide salt is a choline chloride.

4. The method of claim 2, wherein the HBD is a urea, oxalic acid, ethylene glycol, levulinic acid, xylitol, D-sorbitol, D-isosorbide, or glycerol.

5. The method of claim 2, wherein the halide salt and the HBD have a molar ratio of HBD to halide salt having a value from about 1 to about 3.

6. The method of claim 2, wherein the DES has a pH of about 0.7 to about 9.5 when measured at a 10% aqueous solution.

7. The method of claim 6, wherein the DES has a pH of about 5 to about 7 when measured at a 10% aqueous solution.

8. The method of claim 1, wherein the DES is bioderived and/or bio-compatible.

9. The method of claim 1, wherein the introducing steps (a) and (b), and optionally the separating step (c), are continuous.

10. The method of claim 1, wherein the method does not comprise (i) a solid-liquid separation step, (ii) an adjustment of the pH level in the one-pot composition, and/or (iii) a dilution, or addition of water or medium, after pretreatment and/or before saccharification and fermentation.

11. The method of claim 1, wherein the (b) introducing step comprises introducing a microbe to the one-pot composition such that the microbe produces a biofuel from the solubilized biomass.

12. The method of claim 11, wherein the microbe is a biofuel producing microorganism.

13. The method of claim 12, wherein the biofuel producing microorganism is a yeast.

14. The method of claim 13, wherein the yeast is a Saccharomyces cerevisiae.

15. The method of claim 11, wherein the biofuel is ethanol.

16. The method of claim 1, wherein the (b) introducing step comprises introducing an enzyme to the one-pot composition such that the enzyme produces a sugar from the solubilized biomass.

17. The method of claim 16, wherein the enzyme is a cellulase.

18. The method of claim 17, wherein the sugar is a glucose or xylose.

19. The method of claim 16, wherein the method produces a yield of sugar that is equal to or more than about 70%.

20. The method of claim 1, wherein the method further comprises extracting lignin from the solubilized biomass.
